(19) United States
(12) Patent Application Publication
(10) Pub. No.: US 2022/0018836 A1
(43) Pub. Date: Jan. 20, 2022

(54) NON-LIVING SURROGATE INDICATORS AND METHODS FOR SANITATION VALIDATION

(71) Applicant: THE REGENTS OF THE UNIVERSITY OF CALIFORNIA, Oakland, CA (US)

(72) Inventors: Nitin Nitin, Davis, CA (US); Mahmoudreza Ovissipour, Davis, CA (US)

(73) Assignee: THE REGENTS OF THE UNIVERSITY OF CALIFORNIA, Oakland, CA (US)

(21) Appl. No.: 17/344,202

(22) Filed: Jun. 10, 2021

Related U.S. Application Data
(63) Continuation of application No. PCT/US2019/066316, filed on Dec. 13, 2019.
(60) Provisional application No. 62/779,247, filed on Dec. 13, 2018.

(51) Int. Cl. G01N 33/569; G01N 21/65; G01J 3/44; G01J 3/10

(52) U.S. Cl. CPC G01N 33/56911; G01J 3/108; G01J 3/44; G01N 21/658

(57) ABSTRACT

Systems, surrogates, indicators and methods for rapid assessment of sanitation processes are provided. Non-living and non-toxic surrogates applied to a platform or encapsulated in a biological material mounted to a platform are exposed to a sanitation process to be evaluated. Responses to sanitation are measured and quantified using FTIR and chemometrics including principal component analysis (PCA), partial least squares regression (PLSR), loading plots and predictive models. An artificial leaf platform with one or more types of surrogates on one surface and an anchor such as an adhesive film on a second surface is described. Surrogate types include nucleic acid, phage, yeast and algae surrogates. Surrogates may also be attached directly or through a polymer to the platform surface. Surrogates may also be encapsulated or attached to the outside of a biological carrier such as a yeast cell that is free or coupled to the platform.

### CROSS-REFERENCE TO RELATED APPLICATIONS

This application claims priority to, and is a 35 U.S.C. § 111(a) continuation of, PCT international application number PCT/US2019/066316 filed on Dec. 13, 2019, incorporated herein by reference in its entirety, which claims priority to, and the benefit of, U.S. provisional patent application Ser. No. 62/779,247 filed on Dec. 13, 2018, incorporated herein by reference in its entirety. Priority is claimed to each of the foregoing applications.

The above-referenced PCT international application was published as PCT International Publication No. WO 2020/123997 A1 on Jun. 18, 2020, which publication is incorporated herein by reference in its entirety.

### STATEMENT REGARDING FEDERALLY SPONSORED RESEARCH OR DEVELOPMENT

This invention was made with Government support under Grant No. 2015-68003-23411 awarded by the U.S. Department of Agriculture/Initiative for Future Agriculture Food (USDA/IFA). The Government has certain rights in the invention.

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to food sanitization and more particularly to a surface sanitization validation system, non-living surrogate indicators and surrogate carrier platforms and methods for rapid verification of contamination amelioration schemes.

### 2. Background Discussion

Microorganisms are biological entities that exist in the environment and they can be beneficial or hazardous to humans and can be transmitted to humans from food and water. Fruits and vegetables are an important part of human diets a significant part of the food supply. In addition, many of foods in U.S. are consumed as raw including fruits, vegetables, and nuts which makes it difficult to reduce the pathogenic bacteria on them.

With increased consumption, there has been significant increase in the number of foodborne illnesses associated with fresh produce. These foodborne illnesses are the result of contamination of fresh produce with various pathogenic bacteria, parasites, and viruses. Nevertheless, the best way to reduce the bacteria on fresh fruits, vegetables, and fresh cut vegetables is still sanitation and washing. During the sanitation of fresh produce, they go through several washing steps which can also increase the risk of cross contamination if the sanitation level is not high enough to kill the bacteria since the sanitation scheme depends on the removal of bacteria from the surface of fresh produce. Monitoring of the success of the sanitation processes is therefore essential to avoiding cross-contamination and safety.

Food contamination by microorganisms may occur at various stages in the food supply chain. Postharvest handling of fresh produce usually involves various cooling and washing steps as well as various mechanical equipment for transportation, storage and packaging of fresh produce. During these handling steps, fresh produce can be contaminated with microbes from wash water or food contact surfaces.

Disinfection of wash water and equipment are essential to the safety and quality of fresh produce. Therefore, monitoring and rapid validation of sanitizer efficacy is critical to providing safe products by the fresh produce industry.

The food industry uses different methods for process validation, including testing and process control. Conventional testing includes sampling and sending the samples to the accredited laboratory for culturing the bacteria, which takes at least 3 to 5 days to have confirmed results. In addition, the sample size is critical can impact the results. Hence, false negative results are one of the concerns with these methods as well as the cost of tests, and the time between sampling and providing the results.

Many producers in the food industry use process control or monitoring to try to verify results. For example, in sanitation, parameters such as pH, sanitizer concentrations, oxidation-reduction potential are measured. However, there are many disadvantages for process control. First of all, it cannot represent the whole system. In addition, since the system is based on the sensors, any issues and offset can provide false results. This method also cannot provide precise information about the bacteria on the surface of the leaves.

Despite significant efforts to develop a rapid monitoring and validation techniques, the current trends in outbreaks associated with fresh produce shows an urgent need for developing rapid validation tool. The current monitoring approaches include standard plate counting, water chemistry based on sanitizer concentration, total organic content, oxidation reduction potential (ORP), turbidity and pH of the aqueous phase. However, these methods are limited in direct assessment of biological damage induced by sanitizers and can be influenced by complexity of the environment, such as fouling of electrodes and the presence of organic matter.

The mode of action of most of the sanitizers including chlorine, hydrogen peroxide and peracetic acid is based on oxidative stress which could be used as an indicator. Previous studies measured the physiological changes in bacterial cell upon exposure to sanitizers and concluded that the physiological markers have potential to validate the sanitation.

Chlorine is one of the most commonly used sanitizers in the fresh produce industry. Due to the high reactivity of chlorine with organic content, it is critical to monitor and ensure adequate levels of free chlorine during postharvest processing. Oxidation-Reduction Potential (ORP) is one of the analytical standards used to characterize the oxidation potential of chlorine in wash water. pH measurement and pH control are also key steps in maintaining the antimicrobial efficacy of chlorine. Hence, the current practices for monitoring the sanitation of wash water in the fresh produce industry are based on measuring free chlorine, ORP and pH.

However, the antimicrobial efficacy of chlorine depends on multiple parameters including temperature, pH, the amount of available free chlorine in the solution, as well as the amount of organic matter and debris in the water. In addition, there are several limitations in using ORP and pH testing for process validation. For example, the sensors for ORP or pH, like any other equipment, need to be calibrated and maintained, for an accurate data collection and monitoring. In particular, the ORP measurements can be influenced by fouling of the electrodes. Prior studies have suggested limitations of predicting microbial water quality based on ORP measurements in wash water samples with high organic content and in the presence of sediments.

In contrast to measuring chemical properties of water, detecting the presence of bacteria in irrigation water using PCR and conventional culturing are commonly used for monitoring water quality during pre-harvest. However, these methods cannot be easily applied for post-harvest monitoring of wash water quality due to the significant lag time in obtaining results (between 2 to 5 days) needed with conventional culturing along with the expense of frequently conducting PCR analysis on wash water samples.

There are some other detection methods that are available. However, there are drawbacks associated with these methods including the limit of detection, background noise from food materials, and the lack of discrimination between dead and live bacteria. For, example, many of these methods are not able to detect bacteria in quantities less than 100 CFU/g.

Accordingly, there is a need for systems and methods for process control and verification tools that can directly assess the reduction of bacteria during a sanitation process. There is also a need for accurate, rapid, and simple methods for process validation to reduce the cost of recalls, and any damage due to an outbreak.

### BRIEF SUMMARY

The present technology generally provides a system and method for rapid process validation and verification based on non-living edible surrogates including nucleic acids, heat-inactivated yeasts, algae, phages, enzymes, and heat resistance-incorporated surrogate. The surrogates are preferably immobilized on the surface of an inorganic safe material platform or encapsulated using biomaterials and the capsule is mounted to the platform. The functionalized surrogate platforms, for example, may be sent to the processing line and exposed to washing and sanitizing or thermal/non-thermal processing.

The chemical changes in that may occur in surrogates from exposure to processing are detected by vibrational spectroscopy and chemometrics at the level of chemical bonds. The chemical changes in surrogates can be matched with the bacterial reduction, sanitizers concentrations, or any other processing parameters, and the chemometrics model will fit them into a predictive model. By providing the satisfying predictive model and regression coefficient, the results will verify the processing. In another embodiment, the artificial leaves are collected by a metal detector and are used for DNA and phage recovery and analysis.

The term “surrogate” is defined herein as organisms, particles, or substances which are used to study and predict the fate of a microorganism in a specific condition. The United States Food and Drug Administration (FDA) defines surrogates as “a non-pathogenic species and strain responding to a particular treatment in a manner equivalent to a pathogenic species and strain.”

The “artificial leaf” or other useful platform is a platform structure that is coated with an effective amount of indicator on the surfaces to accurately verify an applied sanitization process. An “effective amount” of an indicator cell, device, surrogate composition, capsule or compound refers to a nontoxic but sufficient amount of the cell, device, surrogate composition, capsule or compound to provide the desired result. The exact amount required may vary from subject to subject, depending on the species, age, and general condition of the subject, the severity of the disease that is being treated, the particular cell, device, composition, or compound used, its mode of administration, and other routine variables. An appropriate effective amount can be determined by one of ordinary skill in the art using only routine experimentation.

“Chemometric models” used herein may include principal component analysis (PCA), loading plot, partial least square regression (PLSR), and derived prediction models. For example, models were developed for nucleic acid region (1300-900 cm-1) of the phage spectra. PCA analysis reduces a multi-dimensional dataset, while preserving most of the variances. A PCA analysis shows the clusters and describes similarities or differences in multi-variate datasets. The PC-1 which is the first PC, describes the greatest amount of variation, followed by PC-2, and so on. Each PC has its own score which is comprised of the weightings for that particular PC developing the best-fit model for each sample. Loading plots from PCA may be developed to identify spectral bands that makes significant contribution to the total variance.

On the other hand, PLSR is a bilinear regressed analytical method that develops the relationship between spectral features and reference values (e.g. chlorine concentrations or bacterial count). PLSR models can be developed for each treatment individually and can be evaluated in terms of correlation coefficient (r value), latent variables, standard error, and outlier diagnostic. In addition, a calibration PLSR model can be generated, and cross validated (leave-one-out). In addition, based on the PLSR, the predictive model that was developed uses reference data for the (X-axis) (such as the measured chlorine concentrations or bacterial count) and the Y-axis represents the chlorine concentrations or bacterial count predicted from the FTIR spectra. The suitability of the developed PLSR model can be evaluated by determining the regression coefficient (R), root mean square error (RMSE) of calibration, and the RMSE of cross validation.

In one embodiment, DNA oxidation is measured and changes in DNA conformation is evaluated as a surrogate for assessing effectiveness of chlorine in wash water using infrared spectroscopy. DNA was selected as a surrogate based on the understanding that DNA damage in bacterial cells upon exposure to chlorine is one of the key pathways for inactivation of bacteria. Prior studies have demonstrated both DNA cleavage and chemical changes in base pairs are induced by oxidation processes.

Phage also showed strong potential as a surrogate for predicting sanitizers concentrations and bacterial reduction. Phage could be used for predicting the needed concentrations of two common sanitizers such as PAA and chlorine. IR was shown to provide strong spectra from phage. Chemometrics and mathematical modeling enhanced the phage application as a surrogate through predictive models that are developed based on actual data.

The system and methods provide several benefits and capabilities. The methods directly measure oxidative damage on DNA (preferred), protein and/or lipid biomolecules using vibrational spectroscopy. Measure the changes in spectral signature of the biomolecules. The methods utilize the unique vibrational spectral bands that have been identified for measuring oxidative damage to DNA, protein and lipids induced by sanitizers. Changes in protein, particularly enzyme oxidation may also be evaluated using colorimetric or fluorescence measurements in addition to vibrational spectroscopy.

The system and methods also correlate oxidative damage with sanitizer concentration and bacterial inactivation. This correlation provides quantitative assessment of sanitation efficacy. With linear modelling like PCA, it is possible to detect different clusters of DNA that are subject to different levels of chlorine treatment. Based on the statistical correlations, the methods predict the effective sanitizer concentration and bacterial inactivation on food materials, food contact surfaces and wash water. Thus, effectively validating sanitation process.

These unique strengths are based in part on the surrogate composition as well as the length of DNA (more than 250 bp), the selection of enzymes such as catalases and the immobilization of these compositions in encapsulated structures or on surfaces including cell wall particles, like yeast cell wall compositions, or on polymeric coatings such as Chitosan, Polydopamine or on substrates such as anodisc, ZnO and other inorganic substrates.

For example, Chitosan was shown to improve DNA binding on stainless steel and thus decreased the noise of FTIR signal. Machine learning algorithms can also improve the efficiency of the FTIR signal.

The compositions or selected formulations can also be deposited or coated on a food surface or food contact surfaces. These coated or deposited formulations on food surfaces may provide an assessment of sanitation efficacy of the selected food material. Furthermore, the food product may be selected or modified to enable separation of the coated food products.

In addition, the food product mimicking products may be engineered using diverse materials including plastics or 3-D printed using various polymer components. The engineered materials may mimic the water contact properties of food components. The engineered components may have inbuilt properties such as magnetic properties to enable sorting and separation of these components after processing.

The surrogates and sanitization platforms are preferably biocompatible and suitable for use in contact with the tissues of human beings and animals without excessive toxicity, irritation, allergic response, or other problem or complication, commensurate with a reasonable benefit/risk ratio. “Biocompatible” refers to one or more materials that are neither themselves toxic to the host nor degrade (if the material degrades) at a rate that produces monomeric or oligomeric subunits or other byproducts at toxic concentrations in the host.

This technology is envisioned to be part of a block chain concept for food safety.

Kits containing the surrogate compositions are also provided. The kits typically include the surrogate detection material, optional surrogate supports, and a carrier platform coated with the surrogate or optional surrogate supports. One or more carrier platforms may be provided in the kits with different detection surrogates and indication schemes.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic flowchart of one embodiment of the methods of the invention which shows the steps from the beginning (Preparing the non-living surrogate) until obtaining the results from the instrument.

FIG. 2 is a schematic side view of one embodiment of an artificial leaf depicting several ways for attaching biomolecule surrogates according to the invention.

FIG. 3A is a graph of PCA models of In-Liquid-DNA with PC-1 (98%) and PC-2 (1%) components treated with different concentrations of chlorine for 2 min at 4° C. in the region between 1750 to 800 cm⁻¹.

FIG. 3B is a graph of DNA@Anodisc PCA models with PC-1 (91%) and PC-2 (8%) components treated with different concentrations of chlorine for 2 min at 4° C. in the region between 1750 to 800 cm⁻¹.

FIG. 3C is a graph of PCA models of live Escherichia coli with PC-1 (58%) and PC-2 (25%) components describing bacterial DNA oxidation in live E. coli cells treated with different concentrations of chlorine for 2 min at 4° C. in the region between 1750 to 800 cm⁻¹.

FIG. 4A is a graph of loading plots of In-Liquid DNA treated with different concentrations of chlorine for 2 min at 4° C. in the region between 1320 to 900 cm⁻¹ showing spectral variation in PC-1 and PC-2 loading.

FIG. 4B is a graph of loading plots of DNA@Anodisc treated with different concentrations of chlorine for 2 min at 4° C. in the region between 1320 to 900 cm⁻¹ showing spectral variation in PC-1 and PC-2 loading.

FIG. 4C is a graph of loading plots of E. Coli treated with different concentrations of chlorine for 2 min at 4° C. in the region between 1320 to 900 cm⁻¹ showing spectral variation in PC-1 and PC-2 loading.

FIG. 5A is a graph of correlations of measured chlorine concentrations and as calculated by FTIR spectra coupled with PLSR for In-Liquid-DNA.

FIG. 5B is a graph of correlations of measured chlorine concentrations and as calculated by FTIR spectra coupled with PLSR for DNA@Anodisc.

FIG. 5C is a graph of correlations of measured chlorine concentrations and as calculated by FTIR spectra coupled with PLSR for Escherichia coli.

FIG. 6A is a graph of correlations of measured bacterial count as calculated by FTIR spectra coupled with PLSR for In-Liquid-DNA.

FIG. 6B is a graph of correlations of measured bacterial count as calculated by FTIR spectra coupled with PLSR for DNA@Anodisc.

FIG. 6C is a graph of correlations of measured bacterial count as calculated by FTIR spectra coupled with PLSR for Escherichia coli.

### DETAILED DESCRIPTION

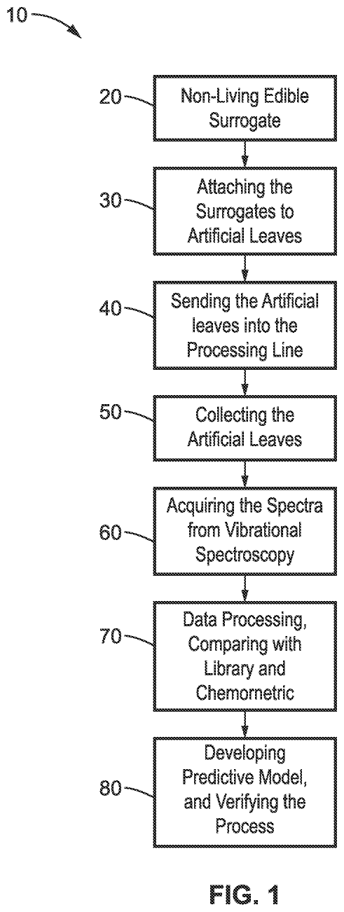

Referring more specifically to the drawings, for illustrative purposes several embodiments of the materials and methods for producing surrogates and artificial leaf platforms for sanitation and process verification are depicted generally in FIG. 1 through FIG. 6C. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

The methods and non-living edible surrogates described herein, have been developed to be used as surrogates for process validation and sanitation verification, along with vibrational spectroscopy and chemometrics. Current methods are dependent on Polymerase Chain Reaction (PCR) to detect the changes in DNA. Based on prior research on pure RNA, DNA, phage and yeast DNA, it was found that the PCR scheme is not able to detect the changes in DNA, particularly when the DNA is short. To overcome the difficulties of PCR, vibrational spectroscopy including Fourier Transform Infra-Red, and Raman are used along with chemometrics and algorithm instead. In addition, FTIR, and Raman spectroscopy will provide comprehensive information about the DNA changes at the level of chemical bonds, including DNA fragmentation, double-stranded to single-stranded conformation, formation of free phosphate groups, deoxyribose changes, and disruption of hydrogen bonds.

None of the attempts in the art for sanitation validation have used naturally existing nucleic acids (RNA, DNA), phages, heat-inactivated yeast, and enzyme, as well as unique surrogate supports including yeast cell wall particles, biomaterials, and FDA approved inorganic substrates, and carrier platforms that mimic the shape, surface and mechanical characteristics of the materials tested.

One preferred method of encapsulation is an impregnation-based system and the surrogate support may be a biological based material with characteristics similar to bacteria in terms of attachment and detachment to the surface.

Turning now to FIG. 1, a flow diagram of one embodiment of a method 10 for the validation of sanitation schemes for inactivation of microbial contaminations is shown schematically. The first step at block 20 of the method of FIG. 1 is the selection of at least one surrogate type and the selected type is thereafter isolated or fabricated. The selected surrogates at block 20 may take several forms that include, but are not limited to, the following:

1. Nucleic Acid Surrogates

In different embodiments, nucleic acids are used as non-living surrogates that are not toxic and can be consumed. Nucleic acids of interest generally include deoxyribonucleic acid (DNA), ribonucleic acid (RNA) from different natural sources. In some embodiments, nucleic acids are selected from microorganisms such as bacteria, yeasts/fungi, algae, or plants and animals without limitation.

2. Yeast Surrogates

Another non-toxic, non-living surrogate type that may be selected at block 20 is heat-inactivated yeast, for example the baker's yeast (Saccharomyces cerevisiae). The cell wall of various yeasts, either live or heat-inactivated, provides similar attachment and detachment properties to bacteria, which makes them appropriate candidates for use as a surrogate. In addition, yeasts are more resistant to physical and chemical stressors such as heat, sanitizers etc. compared to bacteria, which is an important characteristic for use as a surrogate.

In another embodiment, the yeast cells are selected from other yeast groups, including but not limited to, Saccharomyces sp., Candida utilis, Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe and Eremothecium ashbyii.

3. Algae Surrogates

Algae cells are edible microscopic single cell plants, which contains DNA and other cell components. Algae cells which may be selected and used at block 20 include, but are not limited to, Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae and Phaeophyceae (brown algae). In some embodiments, the algal cell is selected from the group consisting of Chlamydomonas reinhardtii, Dunaliella salina, Haematococcus pluvialis, Chlorella vulgaris, Acutodesmus obliquus, and Scenedesmus dimorphus. In some embodiments, the green alga is selected from the group consisting of Chlamydomonas, Dunaliella, Haematococcus, Chlorella, and Scenedesmaceae. In some embodiments, the Chlamydomonas is a Chlamydomonas reinhardtii. In various embodiments the Chlorella is a Chlorella minutissima or a Chlorella sorokiniana cell. Other algal cells of interest include without limitation, Gigartinaceae and Soliericeae of the class Rodophyceae (red seaweed): Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, and Rhodymenis palmate.

4. Phage Surrogates

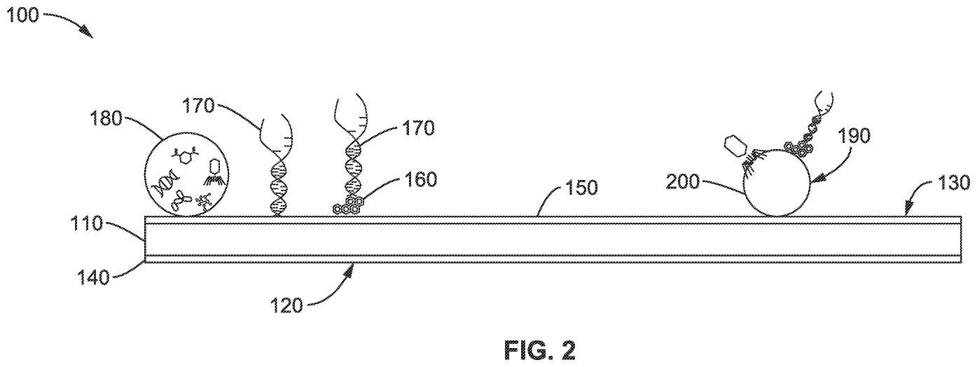

In another embodiment, phages are selected at block 20 and used as surrogates. Phages are listed and generally recognized as safe by the FDA and used in the food industry. These phages include, but not limited to, all members of Siphoviridae and Myoviridae, philBB-PAA2, CEB1, T7, T4, P100, DT1, DT6, e11/2, e4/1c, pp01, 29C, Cj6, F01-E2, A511 phages, can be used as surrogates. In other embodiment, all the FDA approved phages for different bacteria including but not limited to Escherichia coli O157:H7, Salmonella, Listeria monocytogenes, Campylobacter sp., Bacillus sp., Mycobacterium tuberculosis, Pseudomonas sp., Enterococcus faecium, Vibrio sp., Staphylococcus sp., Streptococcus sp., Clostridium sp. Acinetobacter baumannii, are used. Phages are unique and can be used as free surrogates, immobilized on surfaces, or encapsulated in a yeast cell wall or other biomaterials as shown in FIG. 2, for example.

5. Enzyme Surrogates

In another embodiment, the biomaterials that are selected and used at block 20 as non-living, non-toxic surrogates include but are not limited to different enzymes such as superoxide dismutase (SOD), glutathione peroxidase (GPX), catalase (CAT) enzymes which naturally exist in all organism and are responsible for protecting the cells from reactive oxygen species such as ozone, hydrogen peroxide, or other oxidizing agents like chlorine. In the other embodiment, the structural changes and molecular conformation of these enzymes in response to sanitizers could be studied by vibrational spectroscopy and be used as non-living edible surrogate. In another embodiment, the enzymes can be immobilized on a surface of an artificial leaf or encapsulated in yeast cell wall particles or other suitable biomaterials.

6. Cultured Animal of Insect Cell Surrogates

Surrogates selected at block 20 may also be cultured animal cells, insect cells or plant cells. For example, cultured animal cell surrogates may originate from edible animals such as beef, lamb, pork, and seafood including shrimp, fish, and shellfish. Cell based surrogates selected for use at block 20 may also be non-toxic insect cells such as cells from Black Soldier Fly, Grasshoppers, Crickets, Locusts, and Beetles. Surrogates that are selected may also cellular organisms such as bacteria.

7. Heat Resistance Surrogates

In another embodiment, a non-living heat resistance surrogate is developed for thermal processing validation. The surrogate that is selected may be a natural or artificial heat resistant chemical. For example, a natural chemical which is responsible for the heat resistance of certain bacterial spores is dipicolinic acid (pyridine-2,6-dicarboxylic acid or PDC and DPA), which composes 5% to 15% of dry weight of all bacterial spores, may be used. In another embodiment, the DPA can be added to yeast cell wall particles or encapsulated by other biomaterials.

8. Protected Surrogates

In one embodiment, the selected surrogates are protected by molecules such as proteins, lipids, carbohydrates and or mixture of these molecules. The surrogates may also be protected by groups such as consisting of DPA i.e. Dipicolinic acid (pyridine-2,6-dicarboxylic acid) and PDC (4H-pyran-2,6-dicarboxylate) and combinations of DPA and PDC.

9. Carriers for Surrogates

One of the most important parts of process validation is the surrogate properties in terms of hydrophobicity, cell integrity, and resistance to the processing as well as attachment and detachment properties. Surrogates should be able to provide stronger or similar attachment properties to target bacteria and should have stronger resistance to processing compared to bacteria.

In another embodiment, the surrogates are immobilized on the surface of a surrogate support or on carrier platform such as an artificial leaf that can be made from a variety of materials. Surrogate supports may be a capsule coupled to the carrier platform that contains surrogates on the interior or exterior of the capsule surrogate support. The capsules can be structures such as liposomes, cell wall particles, ghosts or fabricated non-biological structures.

The carrier platform materials may include but are not limited to an Anodisc membrane (aluminum oxide), a zinc oxide membrane, a graphene membrane, a gold and silver nanoparticle substrates, a silica oxide membrane, Polydimethylsiloxane (PDMS), chitosan films, alginate films, Poly lactic acid films, Poly ethylene glycol (PEG), Poly diallyl dimethyl amine, Polyvinyl alcohol, Poly (4-vinylpyridine), Poly styrenesulfonate, Poly (maleic acid-co-olefin), Poly dimethylamine, Polyacrylic acid, Polyacrylamide, Poly aspartic acid, Diphosphate, Poly ethylenimine, Oleic acid, Dextran-sulfate, Phosphate-starch, Carboxy methyl dextran.

In another embodiment, surrogates can be encapsulated into the yeast cell wall surrogate support particles, or in other biomaterials including without limitation, carbohydrate polymers such as cellulose, gum Arabic, gum karaya, Mesquite gum, Galactomannans, carrageenan, alginate, xanthan, gellan, dextran, chitosan; proteins such as casein, whey protein, gelatin, gluten, plants protein isolate, plants protein hydrolysates and lipids such as fatty acids/alcohol, glycerides, waxes, and phospholipids.

In order to conduct the process validation, the surrogates may be attached to a carrier platform such as an artificial leaf at block 30 of FIG. 1. In one embodiment, carrier platforms are made from metal and are recoverable at the end of the processing by a magnetic field and metal detector. The attachment and detachment properties of the preferably edible sensors should be comparable with that of real bacteria.

Referring also to FIG. 2, the surrogates may be immobilized on a carrier platform substrate 100. The carrier platform can made from different materials or have a surface layer of a material, including but not limited to an Anodisc membrane (aluminum oxide), zinc oxide membrane, graphene membrane, lignocellulosic materials, gold and silver nanoparticle substrates, silica oxide membrane, Polydimethylsiloxane (PDMS), chitosan films, alginate films, Poly lactic acid films, Poly ethylene glycol (PEG), Poly diallyl dimethyl amine, Polyvinyl alcohol, Poly (4-vinylpyridine), Poly styrenesulfonate, Poly (maleic acid-co-olefin), Poly dimethylamine, Polyacrylic acid, Polyacrylamide, Poly aspartic acid, Diphosphate, Poly ethylenimine, Oleic acid, whey protein, plant based protein, Dextran-sulfate, Phosphate-starch, Carboxy methyl dextran.

In one embodiment, the carrier platform 100 has a flexible body or a ridged body 110 with a bottom surface 120 with attachment points for coupling the carrier platform 100 to a test bed and a top surface 130 for coupling surrogates to the body of the platform or surrogate supports and coupling molecules to the body 110 of the platform 100.

The carrier platform 100 used at block 30 may have different forms based on the plant preference and sanitization processing methods. In some preferred embodiments, the carrier platform body 110 may be flexible with the lower surface 120 that is sticky with a layer 140 on the bottom surface 120 as shown in FIG. 2. The sticky lower layer 140 is used to attach to the platform to surfaces of fruits and fresh produce, for example. The attachment layer 140 could alternatively be magnetic and could be sent through the sanitization system and recovered by the application of a magnetic field.

The carrier platform body 100 may also be fabricated in different shapes, including spherical, flat, tetrahedral, cubic, octahedral, dodecahedral and icosahedral etc. In another embodiment, the carrier platform may have a surface architecture that mimics the surface features and mechanical properties of the meat or food contact surface such as an artificial lettuce leaf.

The surrogate carrier platform 100 may also have different colors to help differentiate them from fruits and vegetables for easy recovery. The carrier platform 100 could also have surface shapes that are similar to those of fruits or vegetables, but in different colors for easy identification. For example, platforms may be provided that are the size of a tennis ball for the apple industry, or the shape of a leaf for the fresh produce industry.

Paper-based carriers 100 such as artificial leaves may have a sticky side 140 which gives them the capability of attaching to fruits, vegetables, and other contact surfaces. The magnetic-based carrier platforms may have a metal core which is covered by natural polymers which gives this embodiment the capability of being recovered at the end of the processing by exposure to a magnetic field.

The surrogates can also be attached to the surfaces of the carrier platforms or surrogate supports in many different ways at block 30. The surrogates can be absorbed, adsorbed, mixed, bound directly or through coupling polymers or carriers. In the embodiment shown in FIG. 2, the top surface 130 of the carrier platform body 110 may have a layer of a polymer film 150. In one embodiment, the surrogates may be attached directly to the polymer film 150 or attached to a coupling polymer 160 that is attached to the top surface 130 or film 150.

Preferred polymers for the polymer film 150 or coupling polymer 160 include polymers such as Polydimethylsiloxane (PDMS), chitosan, alginate, Poly lactic acid, Poly ethylene glycol (PEG), Poly diallyl dimethyl amine, Polyvinyl alcohol, Poly (4-vinylpyridine), Poly styrenesulfonate, Poly (maleic acid-co-olefin), Poly dimethylamine, Polyacrylic acid, Polyacrylamide, Poly aspartic acid, Diphosphate, Poly ethylenimine, Oleic acid, Dextran-sulfate, Phosphate-starch, Carboxy methyl dextran. The polymers can also be added by 3D printing on the surface of the paper and magnetic based artificial leaves. DNA coating on the surface using a biopolymer was shown to improve the sensitivity of the sanitation validation.

One or more types of surrogates can also be coupled to the artificial leaf through a surrogate support structure or coupling molecule. For example, the surrogates may be encapsulated in a support capsule 180 that is coupled to the carrier platform as illustrated in FIG. 2. In another embodiment, the surrogates can be attached to the exterior surface 190 of a surrogate support structure 200. The surrogate may also be attached to exterior surface 190 of the support structure 200 with a coupling polymer.

After producing the functionalized carrier platforms with attached surrogates at block 30 of FIG. 1, the carrier platforms 100 can be introduced into the system where the raw materials are processed and sanitized at block 40. After the previously spiked artificial leaf platforms with surrogates go through the system and are exposed to the processing (sanitation, thermal processing, etc.) similar to the bacteria, the carrier platforms (e.g. artificial leaves) are then collected at the end of the processing at block 50 of FIG. 1.

The exposed carrier platforms that are collected at the end of the sanitation processing are then examined at block 60 and the nature and amount of changes to the surrogates are preferably determined by vibrational spectroscopy and chemometrics.

Generally, the spectra of the exposed surrogates are obtained at block 60 and then processed at block 70 to quantify changes in the surrogates arising from exposure to the sanitization scheme and the changes are correlated to bacterial reduction, for example. This processing at block 70 may also include comparing the spectra with a library of spectra and/or compiled chemometric data.

Vibrational spectroscopy at block 60 may be used to study the mechanism of bacterial inactivation using UV, and chemicals, and allows the development of a chemometric platform for the identification of changes in the samples. It has been observed that the inactivation is a phenomenon involving several mechanisms including cell wall damage, protein and enzymes damage and more importantly, nucleic acid damage, where the amount of damage can be quantified based on chemometric tests and can be correlated to bacterial reduction and the magnitude of the applied stressors. Accordingly, it is possible to detect the changes in non-living surrogates by vibrational spectroscopy and chemometric diagnostics to quantify and develop a predictive model.

In another embodiment, a variety of different vibrational spectroscopy methods that may be used in this invention at block 60 and block 70 include but are not limited to Fourier Transform Infra-Red (FT-IR), Near Infra-Red (NIR), Fourier Transform Near Infra-Red (FT-NIR), Raman, Surface Enhanced Raman Spectroscopy (SERS), Fourier Transform Raman (FT-Raman) and those coupled with microscopes.

After collecting the spectra from the samples at block 60 and processing at block 70, it may be difficult to understand the differences and more chemometrics and mathematical modeling at block 80 may be required for quantification. Since, there are typically some differences among the different readings from samples, the spectra may optionally be pre-processed. Pre-processing may include baseline correction, normalization, and smoothing. In order to provide high resolution spectra, the spectra are preferably processed by second derivative either with Savitzky-Golay or Norris method with different statistical gaps. After this step, still further processing can be applied using, for example, principal component analysis, partial least square regression, prediction model, dendrogram, etc. Partial least square regression can develop the regression between the spectral changes and the magnitude of the processing parameters or bacterial reduction. Based on the model which is developed at block 80, a prediction of the bacterial reduction, or magnitude of the processing parameters magnitude (e.g. chlorine concentration) can be made.

In addition, to reduce the occurrence of a false positive or a false negative, the model may validate the data based by random cross validation or leave-one-out validation. The regression between the predictive model and actual parameters should be more than 0.95% to provide a satisfactory model.

Processing of the acquired spectra preferably includes processing with at least one chemometrics model selected from the group of principal component analysis (PCA), hierarchical cluster analysis (HCA), loading plot, partial least square regression (PLSR), and prediction models. Light GBM is a decision tree algorithm that can improve the predictability of the data to validate sanitation as well as identify key features that improve the discrimination. In addition, other chemometric modeling approaches such as the use of artificial neural networks (ANN), decision trees, supported vector machines and other machine learning tools can be used.

Also, in one embodiment, the pre-processed spectra and second derivative are compared with a big data library that is maintained by a server, and, after matching with the existing data, the results may be provided by the percentage of matching for users, for instance. The library of block 70 can be updated as new or better spectra from analytes are obtained.

The spectra may be collected with either a hand-held or a benchtop instrument, and the spectra may be automatically pre-processed and processed with a computing device with programming. In order to have the final precise results, the spectra from each non-living surrogate, which exposed to a particular processing or sanitizer, should be compared to the reference which has already been provided at block 70 and saved in the cloud or other storage location.

In one embodiment, system users are able to have access to all the reference spectra and updated ones by connecting the instrument to the internet and inserting their user ID and password for downloading the most updated reference library at block 70. System and instrument users can connect to the internet by WiFi and upload the results into the big data library and request for the comparison and receive the results instantly. In addition, for those who do not have access to the internet, the instrument may have its own library incorporated into the instrument. However, the library normally cannot be updated unless the machine connects to internet.

Differentiation is preferably based on the fingerprint of a surrogate. For example, if the surrogate is a nucleic acid-based surrogate the area which is used for data processing, is different from those which are protein based such as enzymes. At the end of the processing, the instrument is able to provide a quantification results with high accuracy, for processing parameter magnitude or bacterial reduction.

The technology described herein may be better understood with reference to the accompanying examples, which are intended for purposes of illustration only and should not be construed as in any sense limiting the scope of the technology described herein as defined in the claims appended hereto.

### Example 1

To demonstrate the operational principles of the methods and surrogates for validating sanitization schemes using chlorine sanitizers, DNA was used as a biochemical surrogate indicator of the success of the process. Structural and chemical changes in DNA molecules that were immobilized on a membrane surface (DNA@Anodisc) and suspended in an aqueous solution (In-Liquid-DNA) were assessed using vibrational spectroscopy and chemometric analysis by a comparison between isolated DNA and the DNA in live Escherichia coli O157:H7 cells. The results of Fourier Transform Infrared (FTIR) illustrated DNA oxidation, fragmentation and conformational changes from double-stranded (ds) to single-stranded (ss) DNA. The PCA model was able to discriminate different groups of samples which were exposed to different concentrations of chlorine (non-lethal, sub-lethal, and lethal; 0, 2, 5, 10, and 15 ppm). PLSR model results showed that the degree of DNA oxidation could be quantified and used successfully to predict the chlorine concentrations and bacterial count. The regression coefficient for predicted vs measured chlorine concentrations and bacterial count were satisfying for all treatments (R²>0.96). The results also showed that the extent of oxidation and fragmentation of DNA was relatively higher for the In-Liquid-DNA, compared to the DNA@Anodisc, and E. coli. The results also suggest that the impact of the chlorine on the DNA@Anodisc and the DNA in the E. coli cells were similar compared to the In-liquid DNA. Overall, the potential of DNA based biochemical surrogate indicator for sanitation process validation of food contact surfaces and fresh produce and demonstrate effectiveness of a chemometric spectral approach for these measurements was validated.

Measuring the chlorine concentration, ORP, and pH in wash water are the main current practices for sanitizing process validation in the fresh produce industry. However, these control parameters cannot provide a direct assessment of bacterial reduction during a washing process. The objective was to demonstrate the development of non-living surrogates for assessing the effectiveness of sanitizers in wash water using vibrational spectroscopy. The results showed that immobilized DNA on anodisc substrates can be used as a non-living surrogate. In addition, vibrational spectroscopy along with chemometric data can be applied for detecting the level of changes in DNA in response to chlorine.

In this example, isolated DNA was suspended in a solution and immobilized on a filter membrane and used to assess oxidative changes induced by chlorine. The results of oxidative DNA damage measured using FTIR was compared with oxidative response of the DNA in a living model bacterium. Salmon sperm DNA was selected as a model isolated DNA and E. coli O157:H7 was selected as a model bacterium. An anodisc filter membrane was selected for immobilization of salmon DNA as the inorganic anodisc membrane does not contribute significantly to the background signal in the FTIR spectral region of the DNA. Immobilization of DNA molecules on anodisc was selected as it could provide an effective approach to introduce and recover DNA molecules in a wash process.

To simulate the washing process conditions, suspended and immobilized DNA molecules were treated with different concentrations of chlorine (2, 5, 10, and 15 ppm) for 2 min. Compositional and structural changes in DNA molecules were assessed using FTIR and the results were compared with changes in the FTIR signature of DNA in live E. coli O157:H7 cells after exposing the cells to the same concentration levels of chlorine for 2 min. The spectral changes in nucleic acid region (1300 to 900 cm⁻¹) were studied using chemometrics to identify key spectral features to detect and differentiative changes in isolated DNA molecules and in DNA of living cells. The results of this study advances understanding for developing DNA based surrogates for process validation in fresh produce industry.

A. Sample Preparation

Solutions with different chlorine concentrations were prepared by dissolving sodium hypochlorite 10% in deionized water to obtain solutions with 2, 5, 10, and 15 ppm of free available chlorine determined via N,N-diethyl-p-phenylenediamine (DPD) colorimetric method. These concentrations were chosen based on previous studies that showed similar ranges can induce non-lethal, sub-lethal and lethal activity in water against E. coli. The pH of the solutions was adjusted for each solution to 6.5 by 0.1 M citric acid. Deionized water with pH of 6.5 was used as a control. In order to mimic the real condition in fresh produce industry, all the DNA samples (i.e. DNA@Anodisc, In-liquid DNA and E. coli O157:H7) were treated at 4° C. with chlorine and stirred using a shaking incubator at 100 rpm speed.

In order to prepare the DNA@Anodisc, 6 mg/ml DNA solution was prepared by dissolving DNA sodium salt from salmon testes (Sigma-Aldrich, St. Louis, Mo.) in sterilized deionized water at room temperature and kept at refrigeration condition for overnight to completely dissolve the DNA. A 50 μl of the stock solution was spotted on top of an anodisc membrane (0.02 mm pore size, 12 mm OD) (Anodisc, Whatman Inc., Clifton, N.J.). Then the DNA@Anodisc was dried under the laminar hood for 2 h to dry the deposited DNA. In order to determine the impact of shear force and water environment on the initial concentration of deposited DNA on anodisc, three DNA@Anodisc samples loaded with 50 μl of 6 mg/ml DNA stock solution, which each anodisc contained 300 μg DNA, were exposed to deionized water at 100 rpm shear force for 2 min at 4° C. The DNA@Anodisc samples were removed and the DNA concentration in water was measured by UV-vis spectrophotometer (GENESYS™ 10S, Thermo Fisher Scientific, Rochester, N.Y., USA) at 260 nm.

The In-Liquid-DNA sample was provided by dissolving the DNA sodium salt from salmon testes (Sigma-Aldrich, St. Louis, Mo.) in sterilized deionized water at room temperature. Then a 50 μl of the stock solution was added to 850 μl of solutions with different chlorine concentrations for 2 min. After 2 min 100 μl of sodium thiosulfate 10% was added to inactivate the chlorine. For the control group, DNA was added to 850 μl of deionized water, and 100 μl sodium thiosulfate was also added to the control group.

Shiga toxin negative Escherichia coli O157:H7 (ATCC 700728, Manassas, Va., USA) was provide by Dr. Linda Harris from the Department of Food Science and Technology at University of California, Davis. The bacteria strain has been modified with a Rifampicin (RIF) resistant plasmid and was cultured on tryptic soy broth (Sigma-Aldrich, St. Louis, Mo., USA) with RIF (50 μg/ml) and grown at 37° C. at 150 rpm. The media was centrifuged at 7,000 rpm for 5 min at room temperature and then the pellet was washed two times with sterile 0.85% saline solution. The pellet then was resuspended in deionized water, and bacterial cells at a concentration of 10⁸ CFU/ml (as determined by standard plating counting method) were prepared and treated with the same levels of chlorine concentration as for the DNA samples. 100 μl of the stock solution was mixed with 800 μl of the chlorine solutions at a specified concentration and vortexed for 2 min. Then 100 μl of 10% sodium thiosulfate was added to stop the reaction.

After chlorine exposure, bacterial concentration in each sample was determined by the standard plate counting method. Briefly, the treated-samples were serially diluted in 0.85% saline solution, spread onto tryptic soy agar plates, and incubated at 37° C. for up to 48 h before enumeration was performed.

B. Fourier Transform Infrared Spectroscopy (FTIR):

For DNA@Anodisc, after each treatment, the DNA@Anodisc were removed and dried under the laminar hood for 2 hours. A 50 μl of In-Liquid-DNA sample from different treatments was spotted on anodisc membranes (0.02 mm pore size, 13 mm OD) and dried under the laminar hood for 2 h. Bacteria were collected using anodisc filter (0.02 mm pore size, 25 mm OD) by filtering 2 ml of the solutions using vacuum filtration. It has been shown that the anodisc membrane filter does not contribute spectral features between the wavenumbers of 4000 and 400 cm⁻¹ and form a relatively uniform thin layer of biological molecules and bacterial cells upon deposition and filtration based on its hydrophilicity.

FTIR spectra were collected using an IRPrestige-21 FTIR spectrometer (Shimadzu Co., Kyoto, Japan). The anodisc filter contained a uniform thin layer of bacterial cells, or DNA was placed in direct contact with the diamond crystal cell of attenuated total reflectance (ATR). FTIR spectra were collected from 4000 to 400 cm⁻¹ at a resolution of 2 cm⁻¹ by adding together 32 interferograms.

C. Data Processing and Chemometrics

Data analysis was performed by Unscrambler® X software (version 10.5) (CAMO Software, Oslo, Norway). Baseline correction was applied to flatten the baseline, followed by normalization. Then the spectra were smoothed with a Gaussian filter of 5 points. In order to reduce overlap in spectral features and to improve discrimination in spectral signatures, second derivative transforms with a gap value of 11 cm⁻¹ using Savitzky-Golay filter were conducted. Chemometric models including principal component analysis (PCA), loading plot, partial least square regression (PLSR), and prediction model were developed for nucleic acid region (1300-900 cm⁻¹). PCA has been used by many researchers for infrared spectra processing and interpretation. PCA reduces a multi-dimensional dataset, while preserve most of the variances. A PCA analysis shows the clusters and describes similarities or differences in multi-variate datasets. The PC1 which is the first PC, describes the greatest amount of variation, followed by PC2, and so on. Each PC has its own score which is comprised of the weightings for that particular PC developing the best-fit model for each sample. Loading plots from PCA were also developed to identify spectral bands that makes significant contribution to the total variance. PLSR is a bilinear regressed analytical method that develops the relationship between spectral features and reference values (e.g. chlorine concentrations or bacterial count). PLSR models were developed for each treatment individually and were evaluated in terms of correlation coefficient (r value), latent variables, standard error, and outlier diagnostic. In addition, the calibration PLSR model was created, and cross validation (leave-one-out) was conducted. In addition, based on the PLSR, the predictive model was developed which the reference data (X-axis) are the measured chlorine concentrations or bacterial count, while the Y-axis represents the chlorine concentrations or bacterial count predicted from the FTIR spectra. The suitability of the developed PLSR model was evaluated by determining the regression coefficient (R), root mean square error (RMSE) of calibration, and the RMSE of cross validation.

D. FTIR Spectral Comparisons

The IR spectra of the salmon sperm DNA suspended in an aqueous solution and the salmon sperm DNA deposited on anodisc filter upon exposure to different concentration levels (2, 5, 10, and 15 ppm) of sodium hypochlorite were obtained. The IR spectra of E. coli O157:H7 exposed to the same set of concentration levels of sodium hypochlorite were taken for comparison.

The IR spectra in each case was acquired between 4000 cm⁻¹ to 400 cm⁻¹. The spectral region between 1300 to 900 cm⁻¹, was selected to assess chemical changes in the DNA induced by sodium hypochlorite. Within this spectral region, the spectral bands at 1051, 1083, and 1230 cm⁻¹ that were assigned to carbonyl deoxyribose stretching vibration, phosphate symmetric and asymmetric vibration, respectively.

The IR spectra results show that overall the intensity of peaks in the spectral region between 1300 to 900 cm⁻¹ for the DNA samples and the bacterial DNA decreased with increasing concentration of sodium hypochlorite till 10 ppm. The peak intensities for both 10 ppm and 15 ppm treatment of the sperm DNA and the bacterial DNA with sodium hypochlorite were similar and show no further decrease in the peak intensities with an increase in sodium hypochlorite concentration from 10 to 15 ppm.

Ratiometric analysis of the specific spectral bands show that the ratio of intensities at 1051 cm⁻¹ with respect to 1083 cm⁻¹ increased with an increase in sodium hypochlorite concentration. In previous studies, this increase in ratio had been attributed to DNA fragmentation.

For 15 ppm treatment of the DNA samples with hypochlorite, the intensity ratio of 1051 cm⁻¹ to 1083 cm⁻¹ increased by 8.5, 5.6 and 5.2 percent for the In-Liquid-DNA, DNA@Anodisc and E. coli O157:H7 cells, respectively compared to the control group, i.e. the untreated samples for each group. In addition, the spectral ratio of peak intensities at 1230 cm⁻¹ to 1083 cm⁻¹ also increased, which was attributed to an increase in single-stranded DNA (ss-DNA), and formation of free phosphate groups, induced by DNA oxidation. For 15 ppm treatment, the spectral ratio increased by 2.1, 6.2, and 5.6% for the In-Liquid-DNA, DNA@Anodisc, and E. coli O157:H7 cells, respectively compared to the control group. These trends also suggest that the influence of chlorine was similar for both the sperm DNA immobilized on anodisc (the DNA@Anodisc) and E. coli O157:H7 cells based on ratiometric measurements with these selected spectral bands.

These results agree with previous observations showing changes in the ratio of bands near 1000 cm⁻¹ (such as ratio of 1016 and 1051 cm⁻¹ to 1083 cm⁻¹). These spectral changes are indicative of oxidative damage to nucleotides in the DNA. Similarly, prior studies have reported changes in the ratio of 1230 to 1083 cm⁻¹ which measures asymmetric/symmetric phosphate band ratio. Increase in the ratio of 1230 to 1083 cm⁻¹ is indicative of DNA damage that results in formation of ss-DNA.

One study of the impact of Fenton's reagent on the Stallion sperm oxidation using FTIR observed that the ratio of the above-mentioned bands could indicate DNA fragmentation and also an increase in ss-DNA formation. Another study of the influence of different concentration levels of hypochlorous acid ranging between 0.025 mM to 0.125 mM on human DNA for 15 min at 37° C. was conducted. This study found that with increasing concentration of hypochlorous acid the bands at 1083 and 1230 cm⁻¹ were shifted by 1-15 cm⁻¹ as a result of an increase in ss-DNA formation. However, in the current study, DNA was exposed to much higher concentrations of chlorine from 2 ppm to 15 ppm for 2 min at 4° C.

The impact of different treatments including chorine on extracellular DNA (16 S rDNA) integrity in solution, bacterial (E. coli) cell viability and genomic DNA has been studied using PCR. It was observed that the disinfection agents such as chlorine, can significantly impact integrity of the pure DNA, but only when applied at higher doses (1000 to 2000 ppm) than those required for E. coli inactivation. It was surmised that the number of sites which are potential targets for chlorine is higher in the genomic DNA than in the 16 S rDNA fragments. In addition, there are many other molecular targets in live cells which can be attacked by chlorine and cause bacterial death without inducing PCR detectable changes in DNA. Their results are different from our findings because of following reasons. In the current study, the extracellular DNA source is from Salmon testes with 30,000 base pair, which compared to 16 S rDNA fragment, is a large DNA with more susceptible sites for reactions with chlorine. In addition, in the current study, vibrational spectroscopy was used that can detect structural and chemical changes in the DNA in contrast to detection of lesions in the DNA using PCR.

E. FTIR Spectra Second Derivative

The results of second derivative of all treatments between 1750 to 850 cm⁻¹ were also obtained and examined. The second derivative reduced replicate sample preparation variability, corrected for baseline shifts, and also resolved overlapping bands. The results showed that in all treatments the peak intensity around 1083 cm⁻¹ was reduced significantly with an increase in chlorine concentration, indicating enhanced DNA strand cleavage and fragmentation with an increase in chlorine concentration.

These results agreed with prior studies that suggest increased DNA strand cleavage in response to oxidative stress generated by sodium hypochlorite treatments. These studies investigated the effect of hypochlorous acid on E. coli genomic DNA to develop a probe for describing the bactericidal action of neutrophils using gel electrography from both in vitro and in vivo studies and extensive genomic DNA fragmentation after exposing E. coli to hypochlorous acid at lethal doses was observed. Other studies of the effect of different disinfectant agents including chlorine on E. coli live cells and E. coli total genomic DNA with PCR reported enhanced DNA fragmentation in cells and extracted DNA after exposing to chlorine.

The second derivative spectra showed that some peaks intensities also increased with an increase in chlorine concentration. The peak intensities around 1035 cm⁻¹ in E. coli, and 1065 cm⁻¹ in the DNA@Anodisc samples increased with an increase in chlorine concentrations. These specific bands are related to C—O stretching ribose. In the DNA@Anodisc samples, the peaks intensity around 1714, 1683, and 1556 cm⁻¹ which are assigned to C═O stretching of guanine, thymine, and adenine, respectively, increased with an increase in chlorine concentration and also shifted by 5 cm⁻¹, in a dose dependent manner upon exposure to chlorine. For the DNA in E. coli cells, similar bands were observed at 1718, 1683, and 1574 cm⁻¹ which were assigned to C═O stretching in guanine, thymine, and adenine, respectively. These trends are similar to the results from a study that has evaluated changes in the calf-thymus DNA conformation after exposing to biogenic polyamines. In contrast to the results with DNA@Anodisc and E. coli samples, the In-Liquid-DNA samples showed only an increase in peak intensity around 1689 cm⁻¹. This peak intensity was assigned to C═O stretching in guanine residues. Previous studies have shown that DNA damage caused by oxidants can result in base lesions, rearrangements, deletions, and insertions. In addition, the DNA In-Liquid, DNA@Anodisc and DNA in cells could be attributed differences in the rate of oxidation reactions of DNA bases in water compared to compacted DNA in cells or DNA molecules adsorbed on surfaces. Furthermore, the presence of excess water can also aid in generation of hydroxyl radicals that may further react rapidly with specific bases on DNA molecules. Overall, the trends agreed with prior studies evaluating DNA oxidation using diverse oxidants.

Others studied the radiation-induced structural modification in dsDNA using FT-Raman spectroscopy and found that by increasing the radiation dose, the guanine and adenine peaks intensity increased. The impact of biogenic amines and Cobalt(III)hexamine on DNA was studied using FTIR, and the results of this study showed that the C═O peaks related to guanine, adenine, and thymine increased with an increase in concentration of biogenic amines and Cobalt(III)hexamine.

Studies of human DNA conformation after exposure to hypochlorous acid found that hypochlorous acid increased the spectral band intensity around 1714 cm⁻¹ in a dose dependent manner. These changes were attributed to guanine oxidation. It has been shown that the reaction of hypochlorous acid with DNA results in both structural and chemical changes, and the heterocyclic NH group of guanine and thymidine derivatives are more reactive and sensitive to oxidation than the exocyclic NH₂ groups. The reaction of chlorine and these heterocyclic groups results in the formation of chloramines which can lead to the ss-DNA formation from ds-DNA due to disruption of hydrogen bonds and formation of nitrogen centered radicals.

F. PCA Models

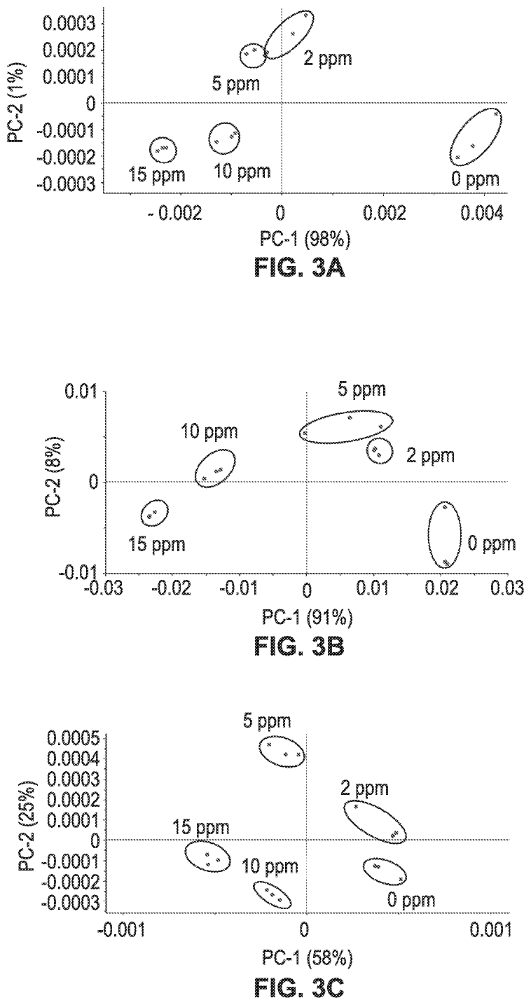

The PCA models for different treatments are presented in FIG. 3A through FIG. 3C. The PCA results show that the spectral changes in DNA induced by sodium hypochlorite is dose-dependent. The PCA model discriminated spectral changes in all the three DNA samples, upon treatment with different concentrations of sodium hypochlorite.

For the In-Liquid-DNA, PC-1 and PC-2 explained 98 and 1%, of variation, respectively, and in the case of DNA@Anodisc, PC-1 and PC-2 components of the PCA model explained 90 and 9% of the variation, respectively. In the case of E. coli, PC-1 and PC-2 components of the PCA model explained 58% and 25% of the variation, respectively. Studies of the impact of Fenton reagent on human sperm DNA damage using Raman and FTIR showed that the PCA model discriminated DNA samples with different level of damages.

Structural changes from exposure of E. coli O157:H7 to different concentrations of chlorine can be evaluated using PCA models. It has been observed that the PC-1 and PC-2, explained 66% and 15% of the variation over the range of 1800 to 900 cm⁻¹ that also included changes in the protein chemical signature. Although the 1300 to 900 cm⁻¹ region was used, the PCA analysis results are similar to the prior studies. In the current study, the PC-1 and PC-2 explained 58% and 25% of the total variation in E. coli which was different from the trend observed with the DNA@Anodisc and In-Liquid-DNA. One possible explanation is that within bacterial cells, chlorine can oxidize different target groups including proteins, enzymes, peptidoglycan, plasmids and genomic DNA and as a result, contributions of PC-1 and PC-2 in the E. coli cellular DNA are different compared to the DNA samples in solution and on anodisc substrates.

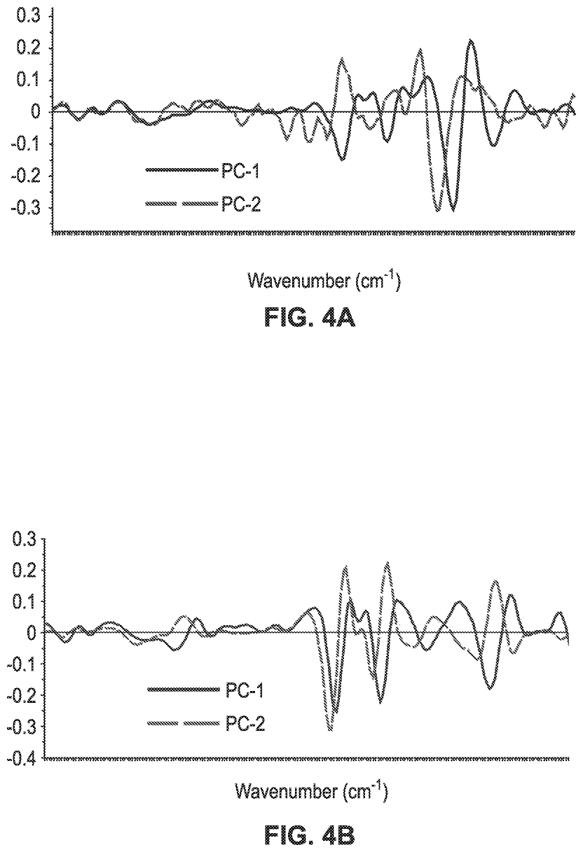

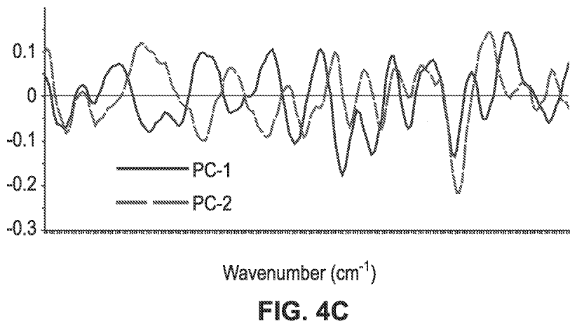

Loading plot of In-liquid-DNA, DNA@Anodisc and Escherichia coli treated with different concentrations of chlorine for 2 min at 4° C. in the region between 1320 to 900 cm⁻¹ are shown in FIG. 4A through FIG. 4C respectively. Loading plots were analyzed to identify the contribution of each key variable (wavenumber) to the principal components 1 and 2. Loading plots can provide a more detailed understanding of the interactions between samples and chlorine and identify significant variables that contribute to spectral changes and associated DNA damage.

The loading plot for the In-Liquid-DNA seen in FIG. 4A showed that the main peak which has the highest contribution to differences in PC-1 was around 1000 cm⁻¹. Changes in the peak around 1000 cm⁻¹ indicate DNA conformational changes associated with an increase in ss-DNA formation.

In the case of, the DNA@Anodisc seen in FIG. 4B and the DNA in E. coli cells seen in FIG. 4C, the loading plots showed that the main band with the highest contribution is 1083 cm⁻¹ for PC1 which is related to symmetric phosphate groups, and for the DNA in E. coli the PC-2 was mainly related to the bands around 1000 cm⁻¹. These results showed that DNA in the liquid phase is more susceptible to oxidative damage by chlorine. In addition, the results showed that the DNA@Anodisc can provide similar changes as observed in the DNA of live E. coli cells and could be used as a surrogate for process validation.

G. PLSR and Prediction Models

PLSR was developed based on the wavenumber between 1300 to 900 cm⁻¹ as x and chlorine concentrations or bacterial count as y. The results for PLSR models for different treatments are presented in Table 1. A good PLSR model should have high values for regression coefficient (R) (>0.95) and low values for RMSE (<1) for calibration and cross validation, as well as reasonable number of latent variables (generally, <10) to avoid overfitting the model (Lu et al., 2011).

The results set forth in Table 1 show that different treatments had reasonable PLSR models based on the regression coefficient and the RMSE values, and the number of latent variables were less than 10 for all the models. In addition, the results showed that both the In-Liquid-DNA and DNA@Anodisc provided promising results for predicting different concentrations of chlorine and the number of bacterial cells. Overall, the In-Liquid-DNA, DNA@Anodisc, and E. coli FTIR spectra, provided similar models and prediction abilities based on R and RMSEs.

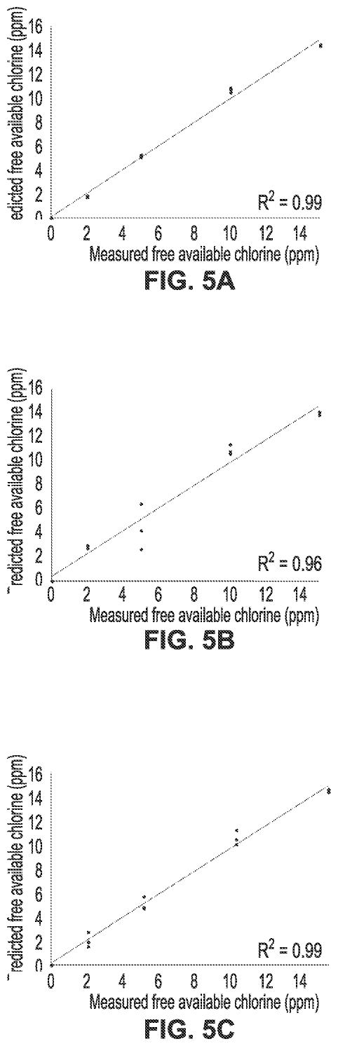

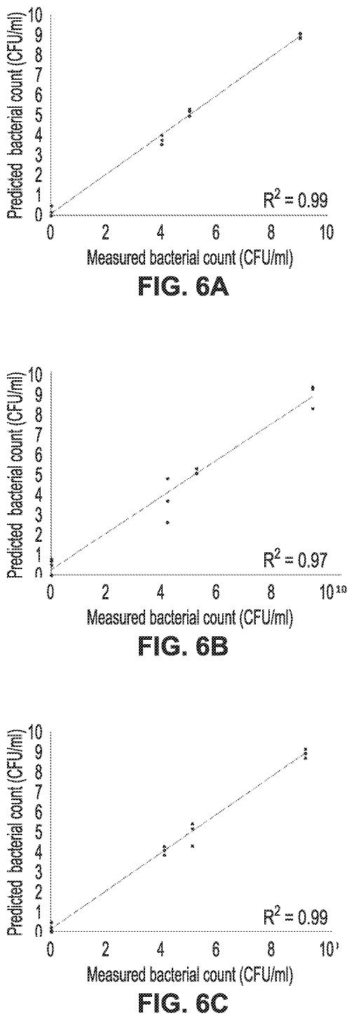

The prediction model results based on the PLS model are presented in FIG. 5A to FIG. 5C and in FIG. 6A to FIG. 6C, representing the chlorine concentrations and bacterial count, respectively. In particular, the correlation of measured chlorine concentrations and those calculated by FTIR spectra coupled with PLSR for In-Liquid-DNA, DNA@Anodisc and Escherichia coli are shown in FIG. 5A through FIG. 5C. The correlation of measured bacterial count and those calculated by FTIR spectra coupled with PLSR for In-Liquid-DNA, DNA@Anodisc and Escherichia coli are shown in FIG. 6A through FIG. 6C.

The results showed strong correlation between the predicted chlorine concentrations or bacterial counts and the actual measured chlorine concentrations or bacterial count. Hence, the results from this study confirm that the PLSR can be used for predicting the chlorine concentration and bacterial reduction based on the FTIR spectra features of the In-Liquid-DNA, DNA@Anodisc, and E. coli.

The results demonstrated that DNA could be used as a biochemical surrogate indicator to assess sanitation process using vibration spectroscopy and to comparing the results with spectroscopic measurements of changes in the DNA of a model bacteria. The results showed that vibrational spectroscopy could be used as a rapid detection method for studying the DNA conformation in response to different concentrations of chlorine. These results suggest that DNA based approaches in combination with chemometric analysis can be used for developing a process control and validation approach for fresh produce sanitation. In addition, the results showed the DNA in bacterial cell and the DNA@Anodisc samples showed similar responses when exposed to different concentrations of chlorine.

Thus, the results indicated that immobilization of the sperm DNA on anodisc membrane may provide a better surrogate to assess response of chlorine on the DNA in bacterial cell than the sperm DNA suspended in a liquid solution.

In addition, the PLSR models can be used for predicting the chlorine concentration and bacterial reduction based on the FTIR spectra features of the DNA upon reaction with chlorine. These models can aid in validation of sanitation processes.

### Example 2

To further demonstrate the methods, the potential of using phage as a surrogate for measuring was evaluated. Phage was used as surrogate for measuring and quantifying phage DNA oxidation and DNA conformational changes in response to sodium hypochlorite and peracetic acid using vibrational spectroscopy.

Bacteriophage was selected as a surrogate due to its abundance in the environment, relatively easy amplification procedures and simple structural compositions (nucleic acid and protein). Upon interaction between phages and sanitizers (e.g. chlorine or peracetic acid), the results of phage DNA oxidation induced by chlorine and peracetic acid were measured and quantified using FTIR and was compared with the oxidative response of the DNA in E. coli O157:H7 as a living model organism and target bacterium.

Measurement and quantification of responses using FTIR and chemometrics verified the surrogates. Chemometrics included principal component analysis (PCA), partial least squares regression (PLSR), loading plots and predictive models. PCA is a well-known unsupervised technique that can reduce the high dimensional data onto lower dimensional space. Partial least square regression is a mathematical model which was successfully applied to develop multivariate calibration models for the vibrational spectroscopy. PLSA used the concentration information (y-data) in determining how the regression factors are computed from the spectral data matrix (x-data) reducing the impact of irrelevant x variations in the calibration model, resulting in more informative data set with reduced dimension and data noise, and more accurate and reproducible calibration models. PLSR was successfully applied for correlating the actual concentrations to spectra and developing predictive models for measuring the concentrations in other settings.

In this demonstration, five phage@anodisc were exposed to PAA at 0, 20, 40, 60 or 80 ppm for 2 min at 4° C. The time and temperature were selected based on food industry sanitation protocol. Similarly, another five phage@anodisc were also exposed to 0, 2, 5, or 10 ppm of chlorine solution at the same conditions. E. coli O157:H7 cells were inoculated into PAA or chlorine following this procedure for 2 min at 4° C. Survivor populations of both phage T7 and E. coli O157:H7 were analyzed to construct survivor plots to evaluate their resistance against sanitizers at selected concentrations.

The survivor population of phage T7 upon treatment with PAA or chlorine at varying levels of sanitizer concentration was shown to be significantly inactivated with an increase of PAA concentration (P<0.05). PAA at 80 ppm concentration successfully inactivated more than 6-log of phage T7. However, despite using high concentration levels, complete inactivation of phages (9 log inoculum level) was not observed. In comparison, complete inactivation of inoculated phages was observed at even the lowest concentration tested (2 ppm of free chlorine). The results suggested that viral particles such as bacteriophages may be more susceptible to chlorine than PAA.

The survivor population of E. coli O157:H7 upon treatment with PAA or chlorine at varying levels of sanitizer concentration was also analyzed. It was observed that E. coli O157:H7 cells were significantly inactivated (2-log inactivation) by PAA, even at 20 ppm. However, no significant inactivation of E. coli O157:H7 was observed with an increase of PAA concentration after 40 ppm. Also, around 4-log survivors were still observed even at the highest levels of PAA (80 ppm for 2 min) used in this study for the initial inoculum levels of 9 log of bacteria.

Phage was selected as a surrogate model for evaluating DNA oxidation after exposure to different concentrations of chlorine or PAA. The major reason that phage T7 are selected as surrogates for evaluating DNA oxidation is that phage T7 is only composed of DNA and protein (capsid), which allows simple FT-IR spectra for data analysis. Chemometrics including PCA, loading plots, PLSR, and predictive models, were developed for the DNA region of the phage@anodisc FTIR spectra from 1300 to 900 cm⁻¹. FTIR spectra were also collected from 4000 to 400 cm⁻¹ at a resolution of 2 cm⁻¹ by adding together 32 interferograms.

The PCA results showed that the spectral changes in the DNA region of a phage@anodisc is dose dependent and PCA model discriminated spectral changes in chlorine or PAA treated phage@anodisc. In the PCA model for PAA treated phage@anodisc, the PC-1 and PC-2 components explained 55 and 26% of the variations in the spectral band corresponding to the DNA region, respectively. In the PCA model of chlorine treated phage@anodisc, the PC-1 and PC-2 components explained 97 and 2% of variations in the spectral band corresponding to the DNA region, respectively.

The PCA results showed that contributions of PC-1 and PC-2 in describing the variations in the DNA spectral bands for PAA or chlorine were similar to the contributions of PC-1 and PC-2 for describing bacterial DNA oxidation in live E. coli cells in the previous example.

Loading plots were prepared to identify contributions of key wavenumbers to the PC-1 and PC-2 analysis. The key wavenumbers identified using the loading plots can aid in understanding biochemical and structural transformation induced in phage DNA upon treatment with PAA or chlorine. Similarly, these results could also be compared with the results of spectral changes in bacterial DNA upon treatment with sanitizers and thus aid in establishing IR measurement of DNA oxidation in immobilized phages as a surrogate for bacterial inactivation.

PLSR models were developed using the 1300 to 900 cm⁻¹ region as x (changes in DNA of phage particles), and chlorine or PAA concentrations or bacterial count as y-axis to develop predictive models for both chlorine or PAA concentrations and bacterial count based on the changes in the spectra. The x-y relationship results explain the contribution of x data, which in this study is related to phage@anodisc wavenumbers, to predict y data, which in this case is related to either sanitizer concentration levels or predicted bacterial count. The results for PLSR models for both chlorine and PAA are presented in Table 2.

An effective PLSR model is expected to have regression coefficient (R) (>0.95), preferably low RMSE (<1) for calibration and cross validation, and reasonable number of latent variables (<10) to prevent overfitting the model. The first four latent variables explained most of the variance (>90%) in PLSR for predicting chlorine or PAA concentrations and bacterial count.

The results of the first latent variable explanation (contribution to predictive models' variances), and x-y relation outliers are presented in Table 3. The results showed that the first latent variable, explained 94%, 82%, 88%, and 98% of the variances for chlorine related concentrations and bacterial count; PAA related concentrations and bacterial count prediction models, respectively. There was a correlation observed between x data contribution and the bands in loading plots. In comparison, the chlorine concentrations of Example 1, only 19% of phage@anodisc DNA wavenumbers (x-data) explained 89% of the chlorine concentrations (y-data) in predictive model. It shows that 81% of the DNA oxidation wavenumbers (x-data) do not contribute significantly in explaining chorine concentrations (y-data). It has been also shown that, wavenumbers that significantly contribute to loading plots appeared as primary factors in latent variable analysis.

The predictive models developed based on PLS, representing the chlorine and PAA concentrations and bacterial count. The results showed strong correlation between predicted sanitizers and bacterial count, and the actual measured sanitizers concentrations and bacterial count, which agrees with the results of Example 1 on pure DNA as a marker for developing predictive models for chlorine concentration and bacterial count.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media or it can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple embodiments which include, but are not limited to, the following:

1. A surface sanitization validation system, the system comprising: (a) one or more surrogate carrier platforms with a top surface and a bottom surface; (b) a plurality of surrogates mounted to the top surface or the bottom surface or the top and bottom surfaces of the carrier platform; (c) a spectral analyzer configured to detect changes in surrogate composition and structure before and after exposure of the surrogates to a sanitization treatment.

2. The system of any preceding or following embodiment, wherein the spectral analyzer is an analyzer selected from the group of Fourier transform IR, Fourier Transform Raman (FT-Raman), Raman, Surface Enhanced Raman and near IR spectroscopes and those coupled with microscopes.

3. The system of any preceding or following embodiment, the system further comprising: (a) a computer processor; and (b) a non-transitory computer-readable memory storing instructions executable by the computer processor; (c) wherein the instructions, when executed by the computer processor, perform steps comprising: (i) acquiring a plurality of vibrational spectroscopy spectra of surrogates on a subject platform; and (ii) pre-processing the acquired spectra with one or more processes selected from the group of baseline correction, smoothing, normalization, and second derivative.

4. The system of any preceding or following embodiment, the instructions further comprising: processing the acquired spectra with a chemometrics model selected from the group of principal component analysis (RCA), hierarchical cluster analysis (HCA), loading plot, partial least square regression (PLSR), and prediction models.

5. The system of any preceding or following embodiment, the computer processor further comprising a transmitter and receiver configured to transmit and receive data to and from a data storage system.

6. The system of any preceding or following embodiment, wherein the carrier platform is made from a material selected from the group of materials consisting of synthetic polymers biopolymers, paper, metals and metal oxides.

7. The system of any preceding or following embodiment, wherein the carrier platform comprises a flexible artificial leaf with a surface that mimics surface features of a natural leaf.

8. The system of any preceding or following embodiment, the carrier platform further comprising a plurality of surrogate supports mounted to the carrier platform, the surrogates coupled to the surrogate supports.

9. The system of any preceding or following embodiment, the surrogate supports comprising a capsule, the surrogates encapsulated within each surrogate support capsule.

10. The system of any preceding or following embodiment, the carrier platform further comprising an adhesive layer applied to the bottom surface of the carrier platform.

11. The system of any preceding or following embodiment, wherein the top surface of the carrier platform further comprises a surface coating selected from the group of coatings consisting of a polymer film, a metal oxide film, a colored film, a magnetic film and a biopolymer film.

12. The system of any preceding or following embodiment, wherein the top surface of the carrier platform further comprises a coating of an anti-oxidant selected from the group consisting of vitamin E, vitamin C, Glutathione, a generic antioxidant and peptides with antioxidative properties.

13. The system of any preceding or following embodiment, wherein the carrier platform has a three-dimensional shape selected from the group of shapes consisting of a sphere, a tetrahedron, a cube, an octahedron, a dodecahedron and an icosahedron.

14. The system of any preceding or following embodiment, wherein the surrogates are selected from the group of surrogates consisting of one or more of DNA, heat-killed yeast, phages, enzymes, RNA, algae, plant cells, insect cells, cultured animal cells bacteria and heat resistant chemicals.

15. The system of any preceding or following embodiment, wherein the enzyme surrogates are enzymes selected from the group consisting of superoxide dismutase (SOD), glutathione peroxidase (GPX) and catalase (CAT).

16. The system of any preceding or following embodiment, wherein the surrogates are protected by groups consisting of DPA, Dipicolinic acid (pyridine-2,6-dicarboxylic acid) PDC (4H-pyran-2,6-dicarboxylate) and a combination of PDC and DPA.

17. The system of any preceding or following embodiment, wherein the heat killed yeast surrogates are selected from the group consisting of Saccharomyces cerevisiae, Saccharomyces sp., Candida utilis, Candida albicans, Candida tropical, Debaryomyces hansenii, Pichia fermentans, Pichia salicaria, Yarrowia lipolytica, Rhodotorula sp. Geotrichum sp., Cryptococcus sp., Lipomyces starkeyi and Phaffia rhodozyma, Fusarium moniliforme, Rhizopus niveus, Rhizopus oryzae, Aspergillus niger, Aspergillus oryzae, Candida guiffiermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe and Eremothecium ashbyii.

18. The system of any preceding or following embodiment, wherein the algae surrogates are selected from the group consisting of Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae, Phaeophyceae (brown algae), Chlamydomonas reinhardtii, Dunaliella sauna, Haematococcus pluvialis, Chlorella vulgaris, Acutodesmus obliquus, Scenedesmus dimorphus, Chlorella minutissima, Chlorella sorokiniana, Gigartinaceae and Soliericeae of the class Rodophyceae (red seaweed), Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, and Rhodymenis palmate.

19. The system of any preceding or following embodiment, wherein the phage surrogates are selected from the group consisting of all members of Siphoviridae and Myoviridae, philBB-PAA2, CEB1, T7, T4, P100, DT1, DT6, e11/2, e4/1c, pp01, 29C, Cj6, F01-E2, A511 phages.

20. The system of any preceding or following embodiment, wherein the phage surrogates are selected from the group consisting of all 2018 FDA approved phages for Escherichia coli O157:H7, Salmonella, Listeria monocytogenes, Campylobacter sp., Bacillus sp., Mycobacterium tuberculosis, Pseudomonas sp., Enterococcus faecium, Vibrio sp., Staphylococcus sp., Streptococcus sp., Clostridium sp., and Acinetobacter baumannii.

21. The system of any preceding or following embodiment, wherein the heat resistant surrogates comprise Dipicolinic acid (pyridine-2,6-dicarboxylic acid) and PDC (4H-pyran-2,6-dicarboxylate) and composes 5% to 15% of dry weight of all bacterial spores.

22. An indicator for a surface sanitization validation system, the indicator comprising: (a) a surrogate carrier platform with an outer surface; and (b) a plurality of one or more types of surrogates mounted to the outer surface of the carrier platform; (c) wherein each type of surrogate produces detectable changes in composition and/or structure of the surrogate with exposure to a sanitization treatment.

23. The indicator of any preceding or following embodiment, wherein the carrier platform further comprises: a bottom surface, the plurality of one or more types of surrogates mounted to a top surface or the bottom surface or the top and bottom surfaces of the platform.

24. The indicator of any preceding or following embodiment, wherein the carrier platform is flexible.

25. The indicator of any preceding or following embodiment, wherein the bottom surface of the carrier platform further comprises an adhesive layer.

26. The indicator of any preceding or following embodiment, wherein the outer surface is coated with a film or a patterned film, the surrogates mounted to the film or patterned film.

27. The indicator of any preceding or following embodiment, the carrier platform further comprising a plurality of surrogate supports mounted to the outer surface of the carrier platform, the surrogates coupled to the surrogate supports.

28. The indicator of any preceding or following embodiment, wherein the surrogate supports of the carrier platform comprise a capsule, the surrogates encapsulated within each surrogate support capsule.

29. The indicator of any preceding or following embodiment, wherein the surrogates are selected from the group of surrogates consisting of one or more of DNA, heat-killed yeast, phages, enzymes, RNA, algae, plant cells, heat resistant chemicals.

30. The indicator of any preceding or following embodiment, wherein the outer surface of the carrier platform further comprises a surface coating selected from the group of coatings consisting of a polymer film, a metal oxide film, a colored film, a magnetic film and a biopolymer film.

31. The indicator of any preceding or following embodiment, wherein the polymer film is a film selected from the group of films consisting of an anodisc membrane (aluminum oxide), a zinc oxide membrane, a graphene membrane, a lignocellulosic material film, gold or silver nanoparticle substrate film, silica oxide membrane, polydimethylsiloxane (PDMS), chitosan films, alginate films, poly(lactic acid) films, poly(ethylene glycol) (PEG), poly (diallyl dimethyl amine), polyvinyl alcohol, poly (4-vinylpyridine), poly(styrenesulfonate), poly(maleic acid-co-olefin), poly(dimethylamine), polyacrylic acid, polyacrylamide, poly aspartic acid, diphosphate, poly(ethylenimine), oleic acid, whey protein, plant based protein, dextran-sulfate, phosphate-starch, and a carboxy methyl dextran film.

32. The indicator of any preceding or following embodiment, wherein the surrogate is bound to the carrier surface with a polymer selected from the group of polymers consisting of polydimethylsiloxane (PDMS), chitosan, alginate, poly(lactic acid), poly(ethylene glycol) (PEG), poly(diallyl dimethyl amine), polyvinyl alcohol, poly (4-vinylpyridine), poly styrenesulfonate, poly (maleic acid-co-olefin), poly(dimethylamine), polyacrylic acid, polyacrylamide, poly aspartic acid, diphosphate, poly ethylenimine, oleic acid, dextran-sulfate, phosphate-starch and carboxy methyl dextran.

33. The indicator of any preceding or following embodiment, wherein the outer surface of the carrier platform further comprises a coating of an anti-oxidant selected from the group consisting of vitamin E, vitamin C, Glutathione, and peptides with antioxidative properties.

34. The indicator of any preceding or following embodiment, wherein the carrier platform has a three-dimensional shape selected from the group of shapes consisting of a sphere, a tetrahedron, a cube, an octahedron, a dodecahedron and an icosahedron.

35. A method for determining the efficacy of a sanitization treatment of a target or targets, the method comprising: (a) selecting a sanitization method for evaluation; (b) providing a surrogate carrier platform with a plurality of one or more types of surrogates mounted to an outer surface of the carrier platform, wherein each type of surrogate produces detectable changes in composition and/or structure of the surrogate with exposure to a selected sanitization method; (c) exposing a collection of one or more carrier platforms and targets to at least one sanitization treatment; (d) acquiring spectra of the treated carrier platforms and surrogates with vibrational spectroscopy; and (e) detecting changes in surrogates from the acquired spectra.

36. The method of any preceding or following embodiment, wherein the spectra is obtained with a spectral analyzer is an analyzer selected from the group of Fourier transform IR, Fourier Transform Raman (FT-Raman), Raman, Surface Enhanced Raman and near IR spectroscopes and those coupled with microscopes.

37. The method of any preceding or following embodiment, further comprising: pre-processing the acquired spectra with one or more processes selected from the group of baseline correction, smoothing, normalization, and second derivative.

38. The method of any preceding or following embodiment, further comprising: processing the acquired spectra with a chemometrics model selected from the group of principal component analysis (PCA), hierarchical cluster analysis (HCA), loading plot, partial least square regression (PLSR), prediction models, neural networks and other deep learning methods.

39. The method of any preceding or following embodiment, further comprising: comparing the acquired spectra with a library of previously acquired and processed spectra; and verifying the efficacy of the sanitization treatment with the comparison.

40. The method of any preceding or following embodiment, wherein the library of previously acquired and processed spectra further comprises spectra of surrogates correlated with known concentrations of sanitizing disinfectants.

41. The method of any preceding or following embodiment, wherein the surrogate is an enzyme selected from the group consisting of superoxide dismutase (SOD), glutathione peroxidase (GPX) and catalase (CAT).

42. The method of any preceding or following embodiment, the carrier platform further comprising: a plurality of capsules mounted to the carrier platform, the surrogates encapsulated within the capsules.

43. The method of any preceding or following embodiment, further comprising measuring a chemical fingerprint of the treated surrogates.

44. A surrogate for use with a surface sanitization validation system, the surrogate comprising: enzymes, known disinfectants or DNA encapsulated in structures such as liposomes or cell wall particles.

45. The surrogate of any preceding or following embodiment, wherein the encapsulated enzyme is an enzyme selected from the group consisting of superoxide dismutase (SOD), glutathione peroxidase (GPX) and catalase (CAT).

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
PLSR Models for Quantification of Chlorine and Bacterial Count

| | Chlorine Concentrations | | | | | Bacterial Count | | | | |
|---|---|---|---|---|---|---|---|---|---|---|
| | | RMSE | | RMSE | No. of | | RMSE | R | RSME | No. of |
| Spectra | R cal | cal | R val | val | latent | R cal | cal | val | val | latent |
| In-Liquid | 0.99 | 0.44 | 0.97 | 1.0 | 4 | 0.96 | 0.23 | 0.98 | 0.4 | 4 |
| DNA | | | | | | | | | | |
| DNA@ | 0.97 | 0.88 | 0.97 | 1.0 | 4 | 0.97 | 0.58 | 0.97 | 0.71 | 3 |
| Anodisc | | | | | | | | | | |
| E. Coli | 0.99 | 0.51 | 0.97 | 0.87 | 6 | 0.99 | 0.32 | 0.97 | 0.60 | 6 |

TABLE 2
PLSR Models for Quantification of Chlorine or PAA and Bacterial Count

| | Sanitizer Concentrations | | | | | Bacterial Count | | | | |
|---|---|---|---|---|---|---|---|---|---|---|
| | | RMSE | | RMSE | No. of | | RMSE | | RSME | No. of |
| Sanitizer | R cal | cal | R val | val | latent | R cal | cal | R val | val | latent |
| Chlorine | 0.97 | 0.61 | 0.95 | 0.82 | 4 | 0.96 | 0.62 | 0.94 | 0.83 | 4 |
| PAA | 0.99 | 1.7 | 0.99 | 2.1 | 4 | 0.99 | 0.16 | 0.99 | 0.17 | 3 |

TABLE 3
The PLS First Latent Variable, x-y Relation Outliers

| | Sanitizer Concentrations | | | Bacterial Count | | |
|---|---|---|---|---|---|---|
| | First Latent Variable | | | First Latent Variable | | |
| Sanitizer | Explanation | x data | y data | Explanation | x data | y data |
| Chlorine | 94% | 19% | 89% | 82% | 40% | 69% |
| PAA | 88% | 39% | 88% | 98% | 36% | 99% |

1. A surface sanitization validation system, the system comprising:
(a) one or more surrogate carrier platforms with a top surface and a bottom surface;
(b) a plurality of surrogates mounted to the top surface or the bottom surface or the top and bottom surfaces of the carrier platform;
(c) a spectral analyzer configured to detect changes in surrogate composition and structure before and after exposure of the surrogates to a sanitization treatment.

2. The system of claim 1, wherein the spectral analyzer is an analyzer selected from the group of Fourier transform IR, Fourier Transform Raman (FT-Raman), Raman, Surface Enhanced Raman and near IR spectroscopes and those coupled with microscopes.

3. The system of claim 1, the system further comprising:
(a) a computer processor; and
(b) a non-transitory computer-readable memory storing instructions executable by the computer processor;
(c) wherein the instructions, when executed by the computer processor, perform steps comprising:
(i) acquiring a plurality of vibrational spectroscopy spectra of surrogates on a subject platform; and
(ii) pre-processing the acquired spectra with one or more processes selected from the group of baseline correction, smoothing, normalization, and second derivative.

4. The system of claim 2, said instructions further comprising:
processing the acquired spectra with a chemometrics model selected from the group of principal component analysis (PCA), hierarchical cluster analysis (HCA), loading plot, partial least square regression (PLSR), and prediction models.

5. The system of claim 3, said computer processor further comprising a transmitter and receiver configured to transmit and receive data to and from a data storage system.

6. The system of claim 1, wherein the carrier platform is made from a material selected from the group of materials consisting of synthetic polymers biopolymers, paper, metals and metal oxides.

7. The system of claim 1, wherein the carrier platform comprises a flexible artificial leaf with a surface that mimics surface features of a natural leaf.

8. The system of claim 1, the carrier platform further comprising:
a plurality of surrogate supports mounted to the carrier platform, said surrogates coupled to the surrogate supports.

9. The system of claim 8, said surrogate supports comprising a capsule, said surrogates encapsulated within each surrogate support capsule.

10. The system of claim 1, the carrier platform further comprising an adhesive layer applied to the bottom surface of said carrier platform.

11. The system of claim 1, wherein the top surface of the carrier platform further comprises a surface coating selected from the group of coatings consisting of a polymer film, a metal oxide film, a colored film, a magnetic film and a biopolymer film.

12. The system of claim 1, wherein the top surface of the carrier platform further comprises a coating of an anti-oxidant selected from the group consisting of vitamin E, vitamin C, Glutathione, and peptides with antioxidative properties.

13. The system of claim 1, wherein the carrier platform has a three-dimensional shape selected from the group of shapes consisting of a sphere, a tetrahedron, a cube, an octahedron, a dodecahedron and an icosahedron.

14. The system of claim 1, wherein the surrogates are selected from the group of surrogates consisting of one or more of DNA, heat-killed yeast, phages, enzymes, RNA, algae, plant cells, insect cells, cultured animal cells, bacteria and heat resistant chemicals.

15. The system of claim 14, wherein the enzyme surrogates are enzymes selected from the group consisting of superoxide dismutase (SOD), glutathione peroxidase (GPX) and catalase (CAT).

16. The system of claim 14, wherein the surrogates are protected by groups consisting of DPA, Dipicolinic acid (pyridine-2,6-dicarboxylic acid), PDC (4H-pyran-2,6-dicarboxylate) and a combination of PDC and DPA.

17. The system of claim 14, wherein the heat killed yeast surrogates are selected from the group consisting of Saccharomyces cerevisiae, Saccharomyces sp., Candida utilis, Candida albicans, Candida tropical, Debaryomyces hansenii, Pichia fermentans, Pichia salicaria, Yarrowia lipolytica, Rhodotorula sp. Geotrichum sp., Cryptococcus sp., Lipomyces starkeyi and Phaffia rhodozyma, Fusarium moniliforme, Rhizopus niveus, Rhizopus oryzae, Aspergillus niger, Aspergillus oryzae, Candida guilliermondii, Candida lipolytica, Candida pseudotropicalis, Mucor pusillus Lindt, Mucor miehei, Rhizomucor miehei, Morteirella vinaceae, Endothia parasitica, Kluyveromyces lactis (previously called Saccharomyces lactis), Kluyveromyces marxianus, Lipomyces starkeyi, Rhodotorula colostri, Rhodotorula dairenensis, Rhodotorula glutinis, Rhodosporium diobovatum, Schizosaccharomyces pombe and Eremothecium ashbyii.

18. The system of claim 14, wherein the algae surrogates are selected from the group consisting of Chlorophyta (green algae), Rhodophyta (red algae), Stramenopiles (heterokonts), Xanthophyceae (yellow-green algae), Glaucocystophyceae (glaucocystophytes), Chlorarachniophyceae (chlorarachniophytes), Euglenida (euglenids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae, Phaeophyceae (brown algae), Chlamydomonas reinhardtii, Dunaliella salina, Haematococcus pluvialis, Chlorella vulgaris, Acutodesmus obliquus, Scenedesmus dimorphus, Chlorella minutissima, Chlorella sorokiniana, Gigartinaceae and Soliericeae of the class Rodophyceae (red seaweed), Chondrus crispus, Chondrus ocellatus, Eucheuma cottonii, Eucheuma spinosum, Gigartina acicularis, Gigartina pistillata, Gigartina radula, Gigartina stellate, Furcellaria fastigiata, Analipus japonicus, Eisenia bicyclis, Hizikia fusiforme, Kjellmaniella gyrata, Laminaria angustata, Laminaria longirruris, Laminaria Longissima, Laminaria ochotensis, Laminaria claustonia, Laminaria saccharina, Laminaria digitata, Laminaria japonica, Macrocystis pyrifera, Petalonia fascia, Scytosiphon lome, Gloiopeltis furcata, Porphyra crispata, Porhyra deutata, Porhyra perforata, Porhyra suborbiculata, Porphyra tenera, and Rhodymenis palmate.

19. The system of claim 14, wherein the phage surrogates are selected from the group consisting of all members of Siphoviridae and Myoviridae, philBB-PAA2, CEB1, T7, T4, P100, DT1, DT6, e11/2, e4/1c, pp01, 29C, Cj6, F01-E2, A511 phages.

20. The system of claim 14, wherein the phage surrogates are selected from the group consisting of all 2018 FDA approved phages for Escherichia coli O157:H7, Salmonella, Listeria monocytogenes, Campylobacter sp., Bacillus sp., Mycobacterium tuberculosis, Pseudomonas sp., Enterococcus faecium, Vibrio sp., Staphylococcus sp., Streptococcus sp., Clostridium sp., and Acinetobacter baumannii.

21. The system of claim 14, wherein the heat resistant surrogates comprise Dipicolinic acid (pyridine-2,6-dicarboxylic acid) and PDC (4H-pyran-2,6-dicarboxylate) and composes 5% to 15% of dry weight of all bacterial spores.

22-42. (canceled)
